(19) United States
(12) Patent Application Publication
(10) Pub. No.: US 2011/0009770 A1
(43) Pub. Date: Jan. 13, 2011

(54) Audiometric Testing and Calibration Devices and Methods

(76) Inventors: Robert H. Margolis, Arden Hills, MN (US); Jonathan D. Birck, Portland, OR (US); George L. Saly, Edina, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 12/834,629

(22) Filed: Jul. 12, 2010

Related U.S. Application Data
(60) Provisional application No. 61/225,090, filed on Jul. 13, 2009.

(51) Int. Cl. A61B 5/00

(52) U.S. Cl. 600/559

(57) ABSTRACT

An audiometric testing device is provided including a housing having one or more integral calibration couplers adapted to couple with a testing transducer. The testing device also includes a tone generator that generates tones of various frequencies and intensities during a hearing test. A calibration transducer is positioned proximate the coupler and converts an output of the testing transducer into a calibration signal. The calibration signal is measured by a signal measurement module within the housing, which generates a calibration measurement that can then be used to correct for undesired intensity level variations produced by the tone generator. Audiometric testing systems and calibration methods are also provided. In some cases a testing and/or calibration of an audiometric testing device is controlled by an external computing device coupled to the testing device.

### CROSS-REFERENCE TO RELATED APPLICATIONS

This application claims priority from U.S. Provisional Application Ser. No. 61/225,090, filed Jul. 13, 2009, and titled “Self-Calibrating Audiometer and Calibration Systems,” the content of which is hereby incorporated by reference in its entirety.

### STATEMENT REGARDING FEDERALLY SPONSORED RESEARCH OR DEVELOPMENT

This invention was made with government support under one or more of grant no. R42 DC007773 and grant no. RC3 DC010986, both awarded by the National Institute on Deafness and Other Communication Disorders, part of the National Institutes for Health. The Government has certain rights in the invention.

### BACKGROUND

Audiometers are well known devices that are used to perform hearing tests. Hearing tests are commonly given in two parts: an air-conduction test and a bone-conduction test. An audiologist gives an air-conduction test and/or a bone-conduction test and the results are displayed on an audiogram. During air-conduction testing, earphones are worn and the sound travels through the air into the ear canal to stimulate the eardrum and then the auditory nerve. The person taking the test is instructed to give some type of response such as raising a finger or hand, pressing a button, pointing to the ear where the sound was received, or saying “yes” to indicate that the sound was heard. The audiometer presents tones at different frequencies (pitches) and at different intensity (loudness) levels.

During bone-conduction testing, a tone is introduced through a small vibrator placed on the temporal bone behind the ear or on the forehead. This method by-passes blockage, such as wax or fluid, in the outer or middle ears and reaches the inner ear through vibration of skull bones. This testing operates in the same manner as the air-conduction testing and is done to measure functionality of the inner ear independent of the functionality of the outer and middle ears. The responses are also recorded on the audiogram. The audiologist then interprets the audiogram.

Audiometers are ordinarily calibrated, thus ensuring accurate and meaningful test results. A number of different electroacoustic features of an audiometer are usually calibrated, including sound pressure level, bone conduction force level, attenuator linearity, frequency, and harmonic distortion.

In some cases, audiometers can be calibrated using an external calibration system, which is often purchased separately at considerable expense and then coupled to the audiometer for calibration. These stand-alone calibration instruments often provide complex routines for calibrating an audiometer that rely on the knowledge and manual intervention of a technician using the calibration system to accurately calibrate an audiometer. Some audiometers have software calibration routines that allow a technician to adjust signal outputs to meet target values specified in audiometer standards. In such cases, a technician can measure numerous sound pressure levels with an instrument such as a sound level meter, and then manually enter the readings into the software routine to adjust signal output levels to match targets.

What is needed is a new audiometer that provides, among other things, a more cost-effective, easier to transport, and easier to use calibration system.

### SUMMARY

Embodiments of the invention are generally directed to novel audiometric testing devices having integrated calibration functionalities and corresponding methods for calibrating an audiometric testing device without the need for stand-alone or single function calibration instruments.

According to one aspect of the invention, an audiometric testing device is provided with a housing that includes a tone output adapted to couple with a lead of a testing transducer and a coupler adapted to receive the testing transducer. A tone generator is positioned within the housing and coupled to the tone output. The tone generator is adapted to generate a plurality of tones, such as in a testing sequence and/or a calibration sequence. A calibration transducer is positioned proximate to the coupler and adapted to convert an output of the testing transducer to an electrical calibration signal. The housing also includes a signal measurement module coupled to the calibration transducer. The signal measurement module is adapted to measure the calibration signal resulting in a calibration measurement. The calibration measurement is useful for, e.g., adjusting operation of the tone generator.

According to another aspect of the invention, an audiometric testing system is provided, including one or more processors, a tone generator coupled with the one or more processors, a tone output coupled to the tone generator, a coupler, a calibration transducer positioned proximate to the coupler, and a signal measurement module coupled to the calibration transducer. The tone generator is adapted to generate a plurality of tones in a testing sequence and a plurality of tones in a calibration sequence based on instructions from the one or more processors. The tone output coupled to the tone generator is adapted to couple with a lead of a testing transducer, and the coupler is adapted to receive the testing transducer. The calibration transducer is adapted to convert an output of the testing transducer to an electrical calibration signal. The signal measurement module is adapted to measure the calibration signal resulting in a calibration measurement and to transmit the calibration measurement to the one or more processors for adjusting operation of the tone generator based on the calibration measurement.

According to another aspect of the invention, a method for calibrating an audiometric testing device is provided. The method includes generating a sequence of tones with the audiometric testing device and outputting the sequence of tones through a testing transducer coupled to the audiometric testing device. The method further includes detecting a sequence of outputs from the testing transducer with the audiometric testing device, the outputs corresponding to the sequence of tones. The method also includes generating calibration signals based on the sequence of outputs with the audiometric testing device and measuring the calibration signals with the audiometric testing device to generate corresponding calibration measurements. In some cases the method also includes adjusting the generation of one or more tones based on the calibration measurements.

Some embodiments of the invention can provide one or more of the following advantages and/or features. Some embodiments include an integrated coupler in the form of an earphone coupler or a bone vibrator coupler, which advantageously allows for calibrating an earphone and/or bone vibrator without the need for dedicated, stand-alone calibration instrumentation. In some embodiments an audiometric testing device is provided as a fully-functioning, stand-alone audiometer including self-calibration capabilities. Some embodiments provide an audiometric testing device capable of being coupled with and controlled by an external computing device (e.g., personal computer). For example, the external computing device can provide a user interface and input mechanisms allowing a user to control the testing device through a software interface provided by the external computing device. In some embodiments, one or more tone outputs are also adapted to couple to the external computing device (e.g., to a sound card), enabling a frequency analysis of one or more of the generated tones by the external computing device. Some embodiments also provide a reference transducer with a known input-output transfer function, which can ensure an accurate calibration of the calibration transducer.

These and various other features and advantages will be apparent from a reading of the following detailed description.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a schematic diagram of a self-calibrating audiometric testing device according to an embodiment of the invention.

FIG. 2 is a high-level schematic diagram of a self-calibrating audiometric testing device according to an embodiment of the invention.

FIG. 3 is a perspective view of an audiometric testing device according to an embodiment of the invention.

FIG. 4 is a side elevation view of the audiometric testing device of FIG. 3.

FIG. 5 is an end elevation view of the audiometric testing device of FIG. 3.

FIG. 6 is a top view of the audiometric testing device of FIG. 3, showing an open coupler configuration.

FIG. 7 is a cross section of FIG. 6 along line A-A.

FIG. 8 is a top view of the audiometric testing device of FIG. 3, showing a closed coupler configuration.

FIG. 9 is a cross section of FIG. 8 along line B-B.

FIGS. 10A-10D are views of a microphone according to an embodiment of the invention.

FIG. 11 is a block diagram of an audiometric testing device according to an embodiment of the invention.

FIG. 12 is a flow diagram illustrating a method for calibrating an audiometric testing device according to an embodiment of the invention.

FIG. 13 is a flow diagram illustrating a method for calibrating an audiometric testing device according to an embodiment of the invention.

FIG. 14 is a flow diagram illustrating a method for calibrating an audiometric testing device according to an embodiment of the invention.

FIG. 15 is a flow diagram illustrating a method for calibrating a calibration transducer within an audiometric testing device according to an embodiment of the invention.

### DETAILED DESCRIPTION OF THE PREFERRED EMBODIMENTS

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

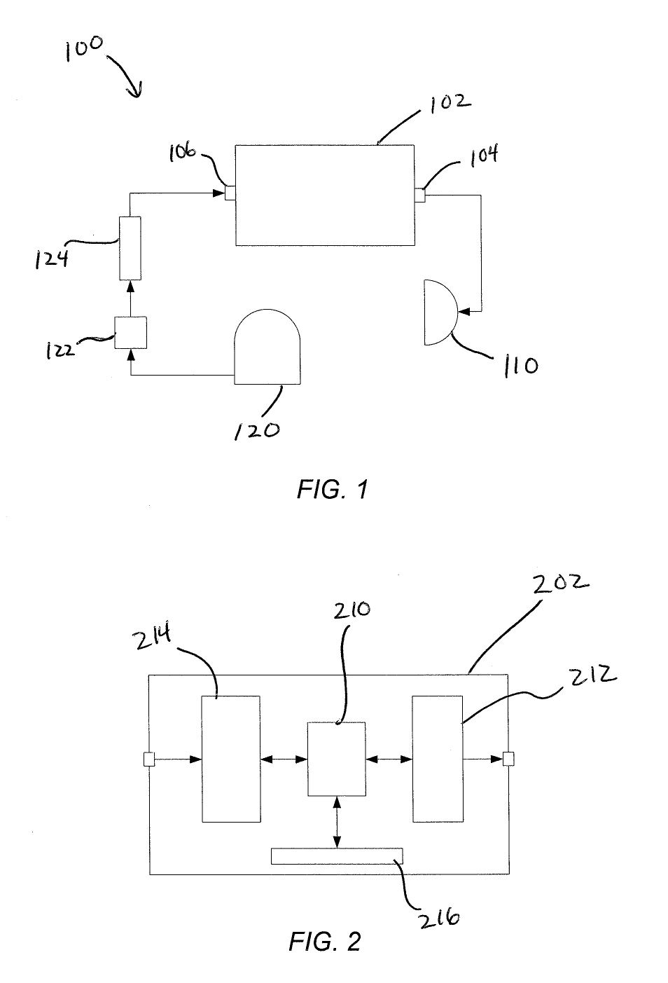

FIG. 1 shows a high-level schematic diagram of an audiometric testing system 100 according to certain embodiments of the invention. The system generally includes an audiometric testing device 102, a testing transducer 110 coupled to a tone output port 104 of the testing device 102, and a calibration coupler 120 coupled to a calibration input port 106 of the testing device 102. According to embodiments of the invention, the audiometric testing system 100 advantageously provides a “self-calibrating” functionality, which will be described in greater detail hereinafter. Among other advantages and features, the calibration functionality allows for calibration of the system 100 without the need for stand-alone calibration instrumentation, or the need for manually measuring and adjusting signal levels required in some past calibration routines.

Returning to FIG. 1, the audiometric testing system 100 includes one or more transducers according to some embodiments. For example, the testing transducer 110 may include one or more earphones for sound conduction testing and/or one or more bone vibrators for bone conduction testing. Those skilled in the art will appreciate that a broad selection of such testing transducers, including earphones and bone vibrators, are commercially available and that the invention is not limited to any particular testing transducer.

The system 100 also includes one or more calibration couplers 120 designed to fit the one or more testing transducers 110. For example, the testing system 100 may be provided with one or more couplers conforming to ANSI, IEC, or other standards for testing audiometer earphone and/or bone vibrator transducers. As just two examples, for calibrating earphones the coupler 120 may be a NBS-9A (ANSI S3.7-1995 (R 2008)) coupler or an IEC 60318 (IEC60318-2-1998) coupler. In some embodiments an artificial mastoid or another such vibrator coupler may be provided for bone vibrator calibration.

According to some embodiments, the calibration coupler 120 is coupled with a microphone 122 and preamplifier 124, and then coupled to the calibration input port 106 of the audiometric testing device 102. FIG. 1 shows this arrangement in schematic form, although those skilled in the art should appreciate that the preamplifier 124, microphone 122, and calibration coupler 120 can be integrated in a number of manners, including being integral with the audiometric testing device 102 (e.g., mounted within a shared housing).

Turning to FIG. 2, in certain embodiments an audiometric testing device 202 generally includes a processor 210 coupled to a sound generation circuit 212 and a calibration circuit 214. The term “coupled” is used herein to indicate that two elements are directly or indirectly connected. As just one example, the sound generation circuit 212 is electrically coupled directly to the processor 210 and indirectly coupled to the calibration circuit 214 through the processor 210.

According to some embodiments, the processor 210 and the sound generation circuit 212 provide commonly known audiometric testing capabilities, typically available in a standard audiometer. For example, the processor 210 is preferably adapted to provide tone generation instructions to the sound generation circuit 212 based on inputs from a technician or test subject. Preferably, the processor 210 also receives calibration measurements from the calibration circuit 214 thus providing a feedback loop during calibration procedures.

In certain embodiments, the testing device 202 also includes a user interface 216 for displaying information to and/or receiving information from an operator. In some cases the user interface 216 is provided with hardware (e.g., input/output devices such as a display, input keys, pointing device, etc.) built into the testing device 202. In some embodiments, the audiometric testing device 202 may be coupled with an external computing device that provides the user interface hardware. It should also be appreciated that testing and/or calibration functionality may be provided by the testing device 202 itself, or alternately shared with or controlled by an external computing device. A wide variety of external computing devices are contemplated, and the scope of the invention is not restricted in this regard. As just a few examples, the external computing device may include a personal computer such as a laptop or desktop computer or a handheld computer (e.g., a PDA, a mobile phone, tablet computer, etc.).

Those skilled in the art will appreciate that audiometric testing devices (e.g., stand-alone and PC-based audiometers) are complex devices and many standard components are omitted from FIG. 2 for clarity. In certain embodiments the testing device 202 may include a wide variety of additional components and features depending upon the desired functionality. For example, the functionality of the testing device 202, including calibration features, may be implemented within a combination of hardware, firmware, and/or software. In addition, audiometric testing capabilities can be provided with hardware configurations having a number of different forms depending upon the desired implementation. In some cases an audiometric testing device is a stand-alone hardware unit. In some cases an audiometric testing device may be configured to be used with a general purpose computer, such as a laptop or desktop computer.

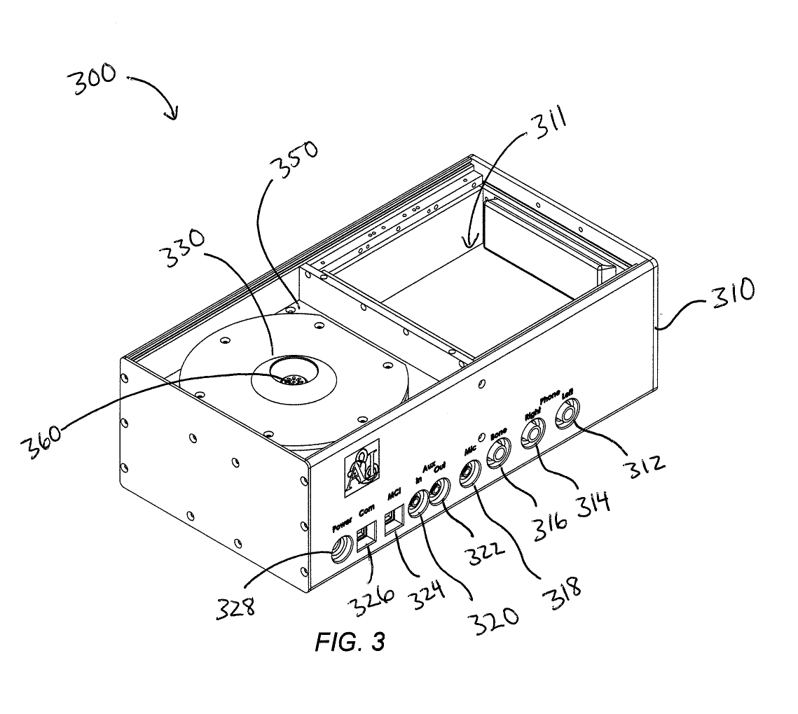

FIG. 3 is a perspective view of an audiometric testing device 300 according to an embodiment of the invention. The testing device 300 includes a housing 310, which in this embodiment includes a sliding drawer 311 providing a convenient location for storing items. As just one example, the drawer may be useful for storing a weight (e.g., a bean bag) useful for compressing together a testing transducer (e.g., such as an earphone) and a transducer coupler. FIG. 5 is an end elevation view of the testing device 300 showing the drawer 311.

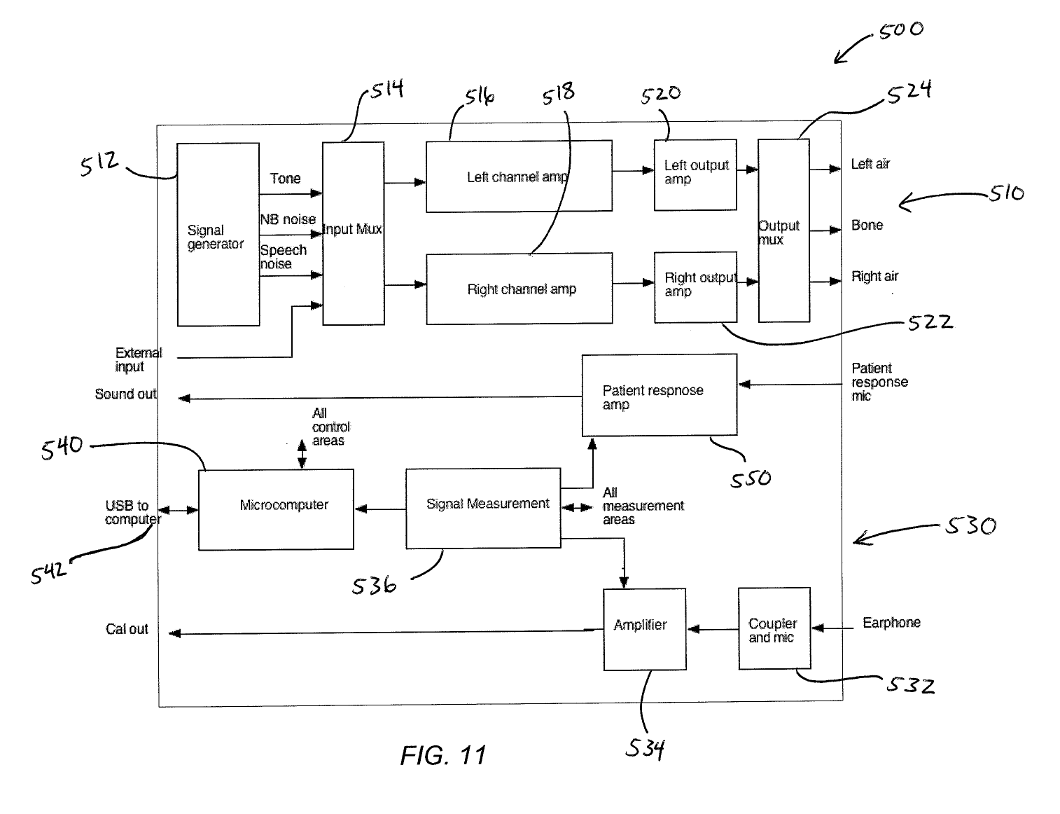

In a preferred embodiment, some or all of the functionality of the audiometric testing device 300 is controlled by a processor within an external computing device (not shown) coupled to the audiometric testing device through the data communication port 326. For example, a personal computer (PC) may be loaded with a software control program which allows a technician to conduct audiometric tests and/or calibration procedures with the audiometric testing device. The PC processor may provide high level control of the testing device, passing instructions to and receiving data from the on-board processor (e.g., see FIG. 11). In some embodiments the PC processor receives calibration measurements from the device, calculates corresponding sound and/or force pressure levels from the testing transducer, determines desired adjustments for calibrating the tone output, and forwards tone generation instructions to the local processor to provide to a tone generator. In addition, the PC processor may control operation of the testing device based at least in part on inputs received through the PC, such as audiometric test and/or calibration sequence commands (e.g., start, stop, pause, etc.), transducer profile information, patient feedback, and other information provided by a person using the PC.

Of course, it is also contemplated that the audiometric testing device 300 may provide all the needed computing functionality needed to perform audiometric testing and/or calibration routines without the need for an external computing device. In such cases, the audiometric testing device may have an onboard processor programmed with instructions for executing such routines.

According to some embodiments of the invention, the audiometric testing device 300 can include one or more inputs and/or outputs for communicating with external components. As shown in FIG. 3, the housing 310 includes left and right tone outputs 312, 314 that are adapted to receive leads of a testing transducer. For example, the left and right tone outputs 312, 314 can comprise jacks adapted to receive corresponding plugs coupled to stereo earphones. The housing 310 also includes an optional bone conduction tone output 316 adapted to couple to the lead of a bone vibrator. Power may be supplied to the testing device 300 through a power input port 328, such as a coaxial power connector providing the device with a DC supply.

According to some embodiments, the testing device includes an optional audio input 318 that can be used to couple a suitable input transducer to the device 300, such as a microphone used during speech audiometry procedures. As shown in FIG. 3, an auxiliary input 320 and output 322 can enable the device 300 to provide additional functions. For example, in some embodiments the auxiliary input 320 is an audio input that can be coupled to an external device, such as a CD or tape player, that presents speech stimuli to the device. In some cases the auxiliary output 322 carries an analog audio output signal (e.g., a signal from the microphone and/or a calibration transducer signal) that can be transmitted to an external computing device for further analysis.

In addition, the audiometric testing device 300 includes two data ports which allow the testing device 300 to communicate (e.g., via digital transmissions) with an external computing device. In some embodiments an audio input port 324 is adapted to couple to a sound card of an external computing device and receive digital audio signals from the external device. For example, the audio input port 324 may be configured as a Media Control Interface (MCI). In a preferred embodiment, the testing device 300 includes a communication port 326, which allows the device to be coupled to and communicate with an external computing device, such as a PC. Thus, a personal computer or other computing device can be coupled with and used to control the testing device 300 through the communication port 326 in order to, e.g., conduct hearing tests.

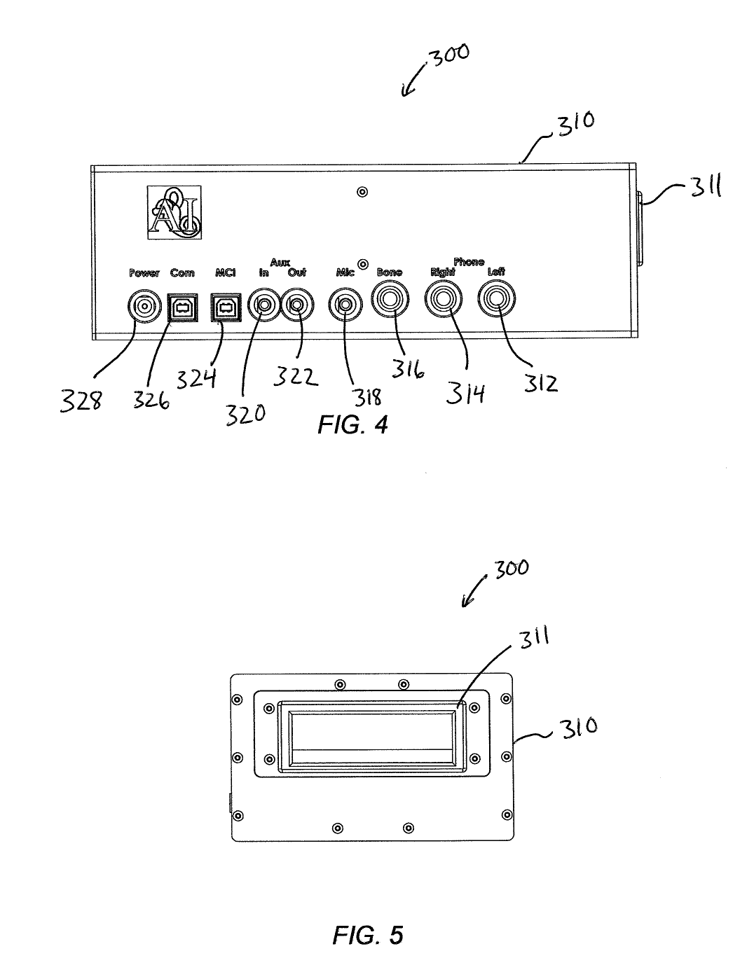

Connections can be made to the testing device using a variety of well-known connectors, and the scope of the invention is not limited in this regard. As just a few examples, in some cases the data ports 324, 326 include Universal Serial Bus (USB) connectors capable of connecting with a USB cable and the left and right tone outputs 312, 314, the bone conduction output 316, the audio input 318, and the auxiliary input and output 320, 322 are phone jacks of a suitable size, e.g., 6.35 mm (¼″), 3.5 mm (approx. ⅛″), 2.5 mm (approx. 3/32″), etc. FIG. 4 is a side elevation view of the testing device 300 providing another view of the inputs and outputs.

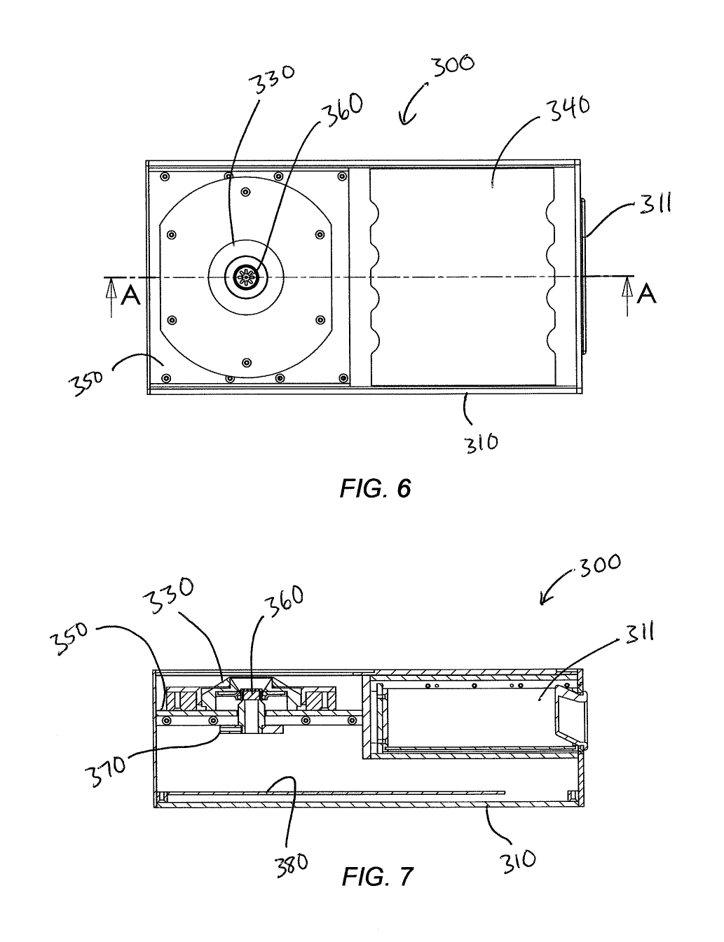

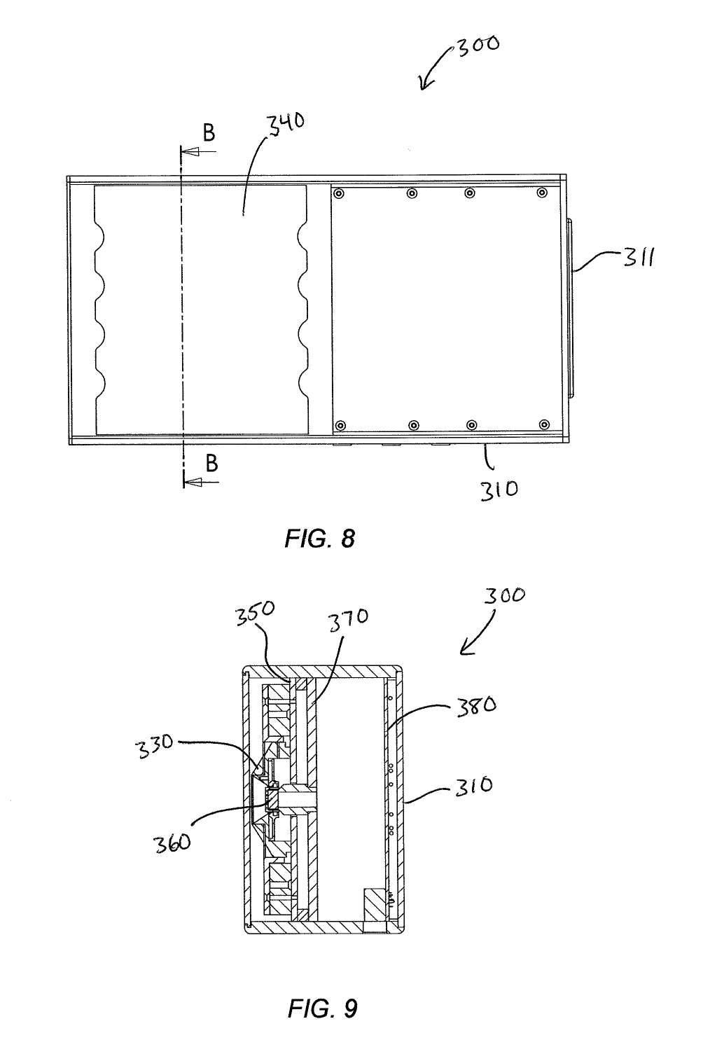

Returning to FIG. 3, in some embodiments the audiometric testing device 300 includes an integrated coupler 330. For example, the coupler 330 may be mounted on or within the housing 310 thus providing a compact and convenient form factor. FIGS. 7 and 9 show cross-sections of FIGS. 6 and 8, respectively, illustrating one possible mounting option for the coupler 330 against a recessed surface 350 of the housing 310. As shown in FIGS. 6 and 8, in some embodiments the housing 310 includes a sliding cover or door 340 that can be moved to alternately reveal and/or conceal and protect the coupler 330. While embodiments are described herein with a single integrated coupler, it is contemplated that two or more couplers (either the same or different) may be included in some embodiments.

As shown in the figures, in certain embodiments the coupler 330 is an earphone coupler, adapted to receive and couple with an earphone, thus allowing the testing device 300 to detect outputs from the earphone. For example, in some embodiments the coupler 330 may be a NBS-9A (ANSI S3.7-1995 (R2008)) coupler or an IEC 60318 (IEC60318-2-1998) coupler. According to some preferred embodiments, the configuration of the coupler 330 corresponds to the left and/or right tone outputs 312, 314 such that the coupler 330 is adapted to receive a testing transducer coupled to one or both of the tone outputs 312, 314. In some cases preferred testing earphone(s) may be packaged with the audiometric testing device 300 to ensure compatibility. Although not shown, in some cases the testing device 300 may also or alternatively include a coupler suitable for mating with a bone vibrator, such as an artificial mastoid.

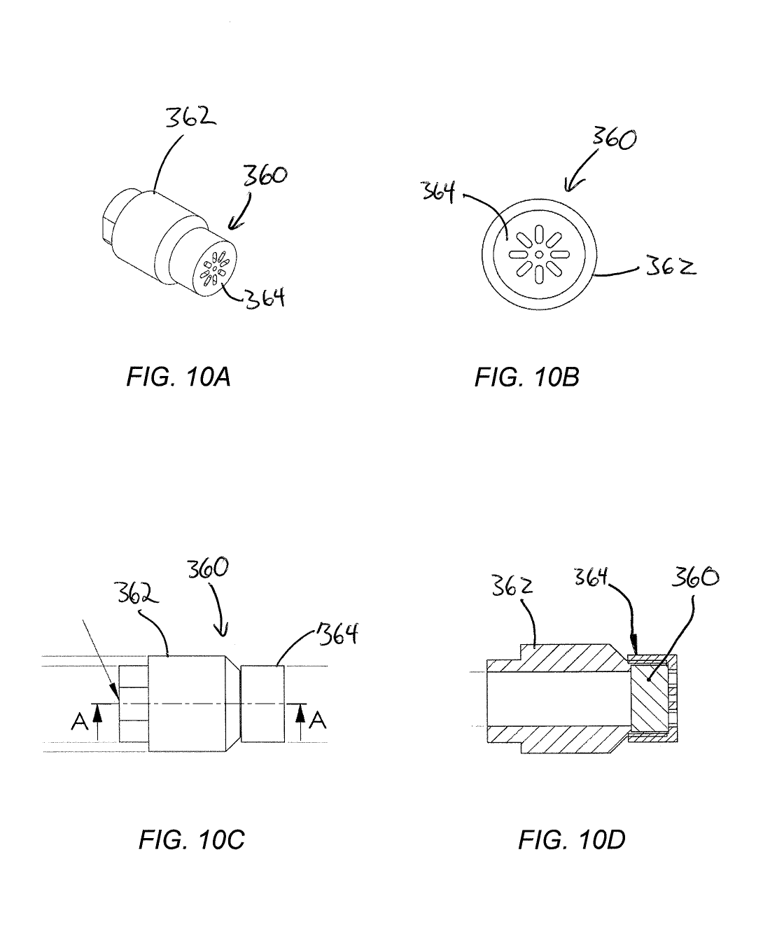

As shown in the figures, in certain embodiments the audiometric testing device 300 includes a calibration transducer 360 positioned proximate to (e.g., mounted within) the calibration coupler 330. In the illustrated embodiment, the calibration transducer 360 includes a microphone adapted to detect and convert an output of a testing transducer placed on the coupler 330 into an electrical calibration signal. FIGS. 10A-10D show multiple views of one example of the microphone 360, including mounting hardware 362 and a protective, screw-on cap 364 provided in some embodiments. According to some embodiments, the microphone 360 can be any suitable acoustic-to-electric transducer or sensor known in the art that converts sound into an electrical signal. In some embodiments the microphone 360 is an electret microphone.

According to a preferred embodiment, the microphone 360 has a frequency response that varies more than 1 dB over a range of selected frequencies. For example, in some embodiments the microphone 360 has a frequency response of between about +/−1 dB and +/−2 dB up to 8 kHz. According to some embodiments, the frequency response is about +/−6 dB up to 8 kHz. Other frequency tolerances, both larger and smaller, are also possible according to embodiments of the invention depending, e.g., upon a particular microphones dynamic range. In some embodiments the microphone 360 may be a more precise transducer element having an approximately flat frequency response, e.g., +/−<1 dB. In some cases a very precise microphone may be used such as those used in sound level meters available from Brüel & Kjær and Larson Davis. However, less precise microphones can also be used in some embodiments, thus avoiding the need for more expensive components.

As shown in FIGS. 7 and 9, in some cases the microphone transducer 360 is mounted upon a support 370 within the housing 310 such that the end of the microphone 360 is positioned within the center of the coupler 330. Although not shown, suitable connections (e.g., wires, traces, etc.) are provided between the microphone 360 and a component circuit board 380 within the housing 310. In some cases the circuit board 380 is a common mounting and connecting platform for the electrical components of the audiometric testing device 300, including calibration and tone generation circuitry (not shown), as well as desired input and/or output connectors.

Turning to FIG. 11, a block diagram illustrating circuitry 500 of an audiometric testing device is shown according to an embodiment of the invention. According to some embodiments, the circuitry 500 generally includes components that generate multiple tones as part of, e.g., a testing and/or calibration sequence. The circuitry also includes components that detect and measure the outputs of a testing transducer, such as a microphone, corresponding to the multiple tones generated.

As shown in FIG. 11, the circuitry includes a tone generator section 510 and a calibration section 530. A processor 540 in the form of a microcomputer is provided to control operation of the tone generator 510 and the calibration circuit 530. According to a preferred embodiment, the processor 540 communicates with an external computing device via a data communication port 542 such as a USB interface (e.g., data port 326 in FIG. 4). The external computing device can be used to control operation of the local processor 540 and the audiometric testing device in general. However, it is contemplated that in some embodiments the processor 540 may include capability to completely control the audiometric testing device without the need for external computing devices. Although not shown in the high level block diagram of FIG. 11, it will be appreciated that the circuitry further includes an internal and/or external power supply (e.g., AC input and AC-DC transformer, batteries, etc.), and may include further components such as onboard memory. In addition, those skilled in the art will appreciate that multiple power and/or data connections between components have been omitted from FIG. 11 for clarity.

According to some embodiments, the tone generator 510 includes components that allow the audiometric testing device to perform functions of a standard audiometer, such as generating pure tones at various frequencies and intensities according to instructions received from the processor 540. In some cases the tone generator 510 may also generate masking noise for masking an ear not being tested and/or speech noise. For example, referring to FIG. 11, in some cases the tone generator 510 generates narrow band (NB) noise and/or speech noise for use during an audiometric test.

In general, the tone generator 510 includes a signal generator 512, such as a tunable oscillator that is capable of generating signals having a range of frequencies. The signal generator 512 is coupled with an input multiplexer 514 that routes one or more distinct inputs into a left channel amplifier 516 and/or a right channel amplifier 518. For example, the input multiplexer 514 may receive several inputs, such as a pure tone, narrow band noise, speech noise, and one or more external inputs. In some embodiments the external inputs are provided via the auxiliary input 320 and/or the audio input port 324 as shown in FIGS. 3 and 4. For example, in some embodiments a CD or tape player or external computer may provide the external input in the form of, e.g., foreign language speech materials or preferred regional materials.

Returning to FIG. 11, the left and the right channel amplifiers 516, 518 are coupled to respective output amplifiers 520, 522, which under the control of the processor 540, can vary the intensity level of a signal to a desired testing level. The output amplifiers are further coupled with an output multiplexer 524 that selects one or more tone outputs, such as left/right air (audio) outputs and/or a bone vibrator output. Upon connecting one or more testing transducers to the tone outputs, pure tones and/or other sounds are converted by the testing transducer(s) to, e.g., sound pressures and/or forces for audiometric testing.

According to some embodiments, the calibration circuit 530 includes a calibration transducer 532, such as a microphone, for detecting and converting the output of a testing transducer to an electrical signal (sometimes referred to herein as a “calibration signal”). The calibration transducer may also be attached or mounted within a transducer coupler as described above. As discussed above, a microphone mounted to an earphone coupler may be used to detect and convert sound pressures generated by an earphone into electrical audio signals. In some cases an artificial mastoid or other such device can be used alone or in conjunction with a microphone to convert forces generated by a bone vibrator into electrical signals. In some embodiments the calibration circuit 530 further includes a preamplifier 534 that amplifies the signal generated by the calibration transducer 532 before passing it for further processing. The preamplifier 534 can include any of a large number of preamplifiers known in the art.

According to some embodiments, the calibration circuit 530 may be coupled to a signal measurement module 536 similar to circuitry within a sound level meter that receives and measures the audio calibration signal from the calibration transducer 532. The signal measurement module 536 may include a number of separate components, or may be implemented partially or wholly using the processor 540. In some embodiments the signal measurement module includes one or more amplifiers, an analog-to-digital converter in the processor 540, and a memory module (not shown). The signal measurement module 536 measures the calibration signal to generate a calibration measurement characterizing the calibration signal and transmits the calibration measurement back to the processor 540 for determining whether calibration adjustments are needed. According to some embodiments, the signal measurement module 536 may also be used for measuring other signals. For example, in some embodiments the signal measurement module 536 is also coupled to other components in the circuitry 500 (e.g., left and right channel amplifiers 516, 518, output amplifiers 520, 522, etc.) to measure intermediate signal levels and provide a feedback control loop.

According to some embodiments, the circuitry 500 of the audiometric testing device also includes a patient response channel that receives, amplifies and passes on patient responses during speech audiometry procedures. For example, in some embodiments patient responses are received from the audio input 318 (FIG. 4) coupled to a patient microphone. The responses are amplified through a patient response amplifier 550, and then transmitted to an external computing device via a sound output port, such as the auxiliary output 322 (FIG. 4).

In certain embodiments, a transfer function of the calibration transducer 532 is stored within an external computing device coupled to the audiometric testing device. According to some embodiments, the external computing device is programmed with instructions for calculating a sound or force pressure level emitted by the testing transducer based on the calibration transducer transfer function and the calibration measurement performed by the signal measurement module 536. The external computing device may be programmed to compare the determined transducer output level directly to a desired output level in order to determine whether and by what amount adjustments in tone generation are needed to generate the tone output from the tone generator in accordance with the desired output levels. For example, in some embodiments the external computing device may compare the measured output level directly to Reference Equivalent Sound Pressure Levels (air conduction) and/or Reference Equivalent Force Levels (bone conduction) that are provided in American (ANSI S3.6-2004) and international (ISO/DIS 389-8-2004) audiometry standards.

Of course, it is also contemplated that the onboard processor 540 may also be programmed with instructions (e.g., firmware and/or software within a memory module independent from and/or integrated with the processor 540, not shown) for calculating the levels emitted by the testing transducer and comparing the calculated levels with a desired output level in order to determine a desired correction factor.

Thus, the degree of deviation from the relevant standard and also a correction factor necessary to bring the output closer to the desired standard output can be determined. The calculated correction factors can then be used in future operation of the audiometric testing device in order to provide a more highly calibrated tone output. According to some embodiments, one or more measurements and/or calculations may be recorded in a calibration history or profile (e.g., in a coupled memory not shown or an external computing device). Correction factors can then be recalled as necessary to adjust operation of the tone generator when generating pure tones during an audiometric test.

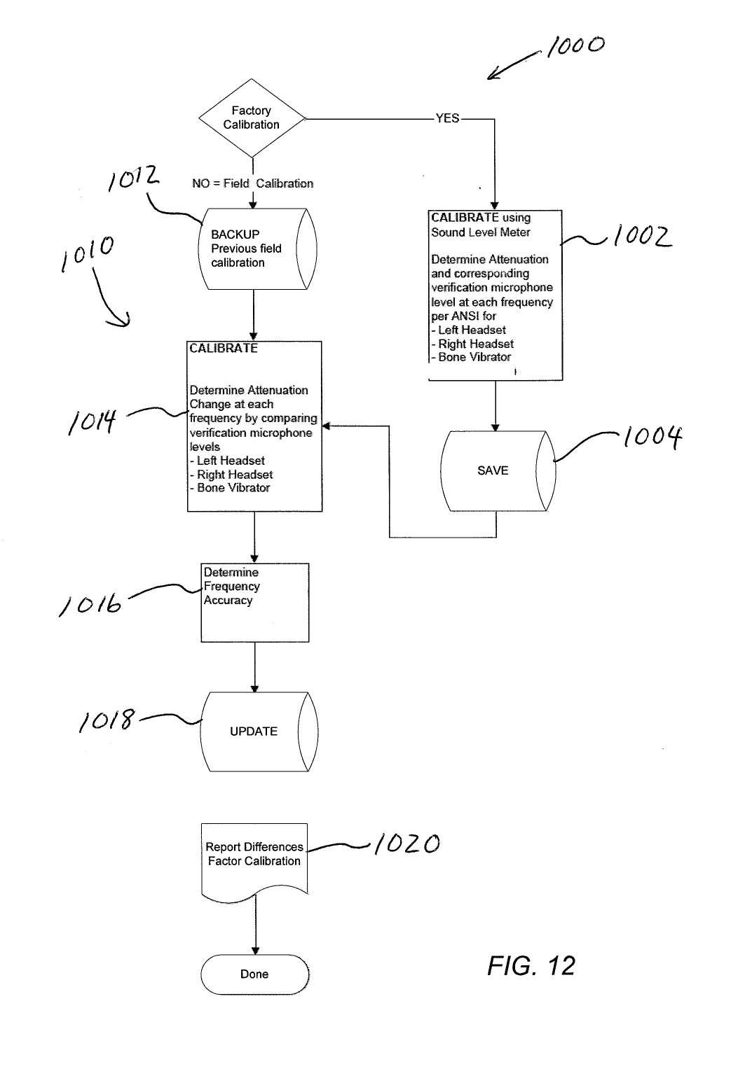

Various methods for calibrating an audiometric testing device will now be discussed with reference to FIGS. 12-15. In some cases an audiometric testing device and/or externally coupled computing device preferably stores one or more audiometric testing routines and one or more calibration routines. For example, the routines may be part of a software and/or firmware control program stored in a non-transitory computer-readable medium such as random access memory or read only memory. It should be appreciated that a wide variety of physical memory forms are possible (e.g., compact disc, digital video disc, solid state memory, memory integral to a microprocessor, etc.) and the scope of the invention is not limited in this respect.

Prior to execution, the software instructions are programmed into one or more processors, such as the microprocessor of an externally coupled PC and/or an on-board processor of the audiometric testing device. When executed, an operator may be able to select one or more testing and/or calibration procedures for the audiometric testing device to perform, and then initiate the procedures by simply pressing a start button (e.g., on an on-board interface and/or a remote PC). Embodiments of the invention thus provide a considerably easier and more convenient way for an operator to calibrate an audiometric testing device than with existing methods that require manual measurement and adjustment of output levels, or the use of expensive and complicated external calibration instruments. In addition, because the calibration functionality is provided within the testing device, calibration can occur at any convenient time and location in which testing may be planned. An operator can further calibrate the audiometric testing device without the need for training on the use of separate calibration systems.

FIG. 12 is a flow diagram 1000 illustrating possible approaches for calibrating an audiometric testing device with one or more testing transducers such as earphones or bone vibrators. In some embodiments a factory calibration 1002 is performed using standard calibration equipment, such as a sound level meter. After determining correction factors for one or more frequencies, the necessary adjustments are saved 1004 in memory for future use. According to certain embodiments, device/transducer calibration can be performed in the field with the audiometric testing device, and without the need for typical calibration equipment such as stand-alone sound level meters. FIG. 12 illustrates one example of a field calibration routine 1010.

In some cases one or more preliminary pre-calibration actions may be performed as part of the field calibration. One or more of the pre-calibration steps may be performed by the audiometric testing device, a coupled external computing device, and/or with the assistance of a technician. For example, the technician may in some cases manually key in or otherwise input the types of transducer and coupler currently installed. In some cases the audiometric testing device may display instructions for guiding the technician through such manual steps. Some calibration routines may include one or more of the following steps:
1. Determine the transducer to be calibrated (bone vibrator/earphone);
2. Determine the earphone to be calibrated (left/right and type);
3. Install the correct coupler for the earphone;
4. Determine the date;
5. Determine the operator; and
6. Load the current calibration table as a starting point for setting the level to be used for calibration.

Referring again to FIG. 12, in some embodiments the most recent calibration settings are backed up 1012 prior to beginning the calibration procedure. The method 1010 preferably includes calibrating one or more testing transducers, such as, for example, left and right earphones (sometimes also referred to as “headsets”) and/or a bone vibrator. In some embodiments the intensity output levels of the testing transducer are calibrated 1014 (e.g., to Reference Equivalent Sound Pressure Levels (air conduction) and/or Reference Equivalent Force Levels per ANSI and/or ISO/DIS audiometry standards) by determining the correction factor (e.g., attenuation change) necessary to more accurately generate tones at one or more frequencies. In certain embodiments, frequency characteristics of the testing transducer output may also be analyzed and calibrated 1016. Upon making the desired adjustments, the new correction factors can be saved to update 1018 the calibration settings. In some cases, the differences between the most recent and the new calibration settings (i.e., reflecting the change in the testing transducer output) are reported 1020 to a technician via a user interface and/or logged to a data file for future reference.

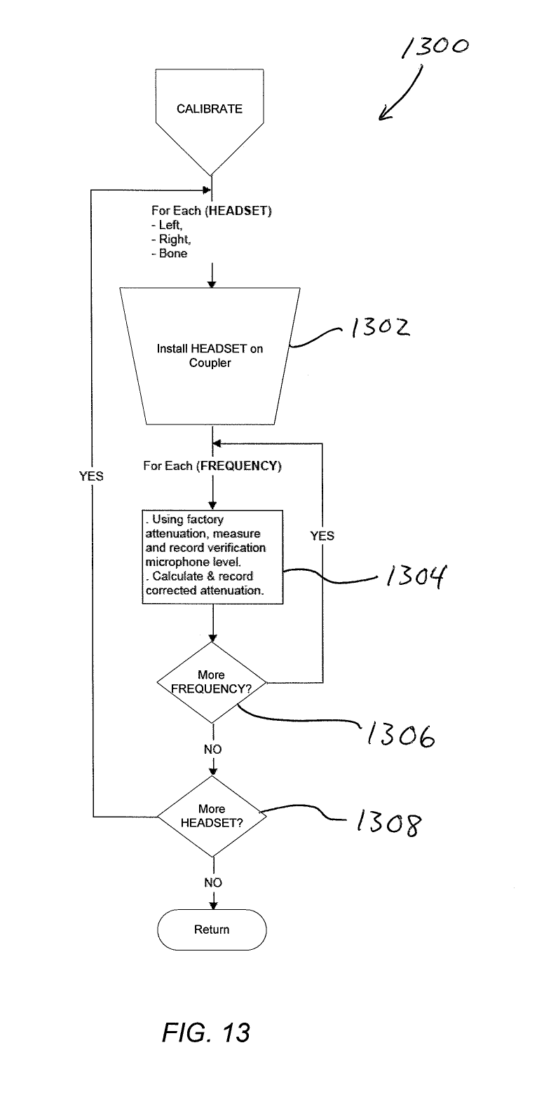

FIG. 13 illustrates a method 1300 for calibrating the testing transducer output intensity levels according to a preferred embodiment. For example, as an initial step, a particular testing transducer (e.g., headset) is selected and installed 1302 on the coupler of the audiometric testing device. It should be appreciated that the method of installation will vary depending upon the type of coupler and transducer. In some embodiments an earphone may be held in place on an earphone coupler by a sufficient weight.

After installing 1302 the testing transducer, the calibration routine 1300 includes generating a sequence of calibration tones to calibrate 1304 the tone generator at one or more frequencies of interest. At each frequency, the tone generator generates a pure tone having a nominal intensity level, and the calibration transducer output is measured and processed to determine if the output of the testing transducer is within a desired tolerance of the desired intensity level. An example procedure according to some embodiments may include the following actions:
1. Set the frequency of the tone to be calibrated;
2. Set the tone level;
3. Turn on the tone;
4. Read the level;
5. Display appropriate information to the user;
6. Adjust the level up or down until the measured level is within the guardband established for calibration tolerance;
7. Turn the tone off; and
8. Save the setting in memory.

In some embodiments the calibration of a single frequency may be performed multiple times in iterative fashion to provide an increasingly accurate output signal. After calibrating a single frequency, the method determines 1306 whether calibration is desired for additional frequencies and the calibration procedure 1304 is repeated if necessary. For example, in some embodiments a sequence of frequencies are calibrated within a specified range. In one preferred embodiment, the following sequence of frequencies in the range between 125 Hz and 8000 Hz are calibrated: 125, 250, 500, 750, 1000, 1500, 2000, 3000, 4000, 6000, and 8000 Hertz.

After calibrating for all the desired frequencies, it is determined 1308 whether calibration is desired for one or more additional testing transducers, and the method can be repeated for each additional transducer. After ending the calibration routine, the method may then prompt the operator to (or may automatically) save the new calibration file in memory and/or generate (e.g., print) a certificate of calibration with appropriate information. Of course, it should be appreciated that this is merely one example of a calibration routine and that one or more steps may be omitted, modified, and/or added depending upon the criteria of a particular embodiment.

According to some embodiments, some, most, or all of the calibration steps 1304 may be executed automatically by one or more processors, such as a processor within a coupled external computing device and/or an on-board processor in the audiometric testing device. Thus, calibration of the testing device and testing transducer can be quickly and conveniently accomplished in a relatively short time with minimal input from a device operator/technician. In some embodiments, calibration of a single earphone may take place in, for example, less than two minutes.

Accordingly, embodiments of the invention can provide a variety of useful calibration functionality previously unavailable in an audiometric testing device. For example, calibration can be performed at any time without the aid of a stand-alone calibration system. An operator can, for example, easily schedule periodic preventative calibrations more frequently than customary annual calibrations. In addition, in the event of unexpected or doubtful test results, an operator can confirm the calibration of the audiometric testing device directly after testing to check the accuracy of the previous test results.

According to some embodiments, a method of calibration may request and/or accept one or more data inputs from an operator. For example, in some cases a user interface may request one or more of the following inputs from a technician operating the testing device:
1. Set of tones of various frequencies to be calibrated;
2. Type of transducer to be calibrated;
3. Whether the earphone is left or right side;
4. Conversion factor from sound pressure level (SPL) to hearing level (HL) for frequencies used in the calibration;
5. Level at which calibration is to be performed;
6. Name of operator performing the calibration; and/or
7. Date of the last calibration.

In some cases the calibration method may generate one or more outputs for review by an operator. For example, in some cases some or all of the following outputs may be logged and transmitted, printed, and/or displayed on a user interface:
1. Current frequency of the tone being calibrated;
2. Target level in SPL;
3. Currently measured SPL;
4. Old and new calibration value illustrating difference in calibration;
5. Option to accept/reject/save the new values;
6. Date of current calibration; and/or
7. One or more of inputs entered into the system, such as those noted above.

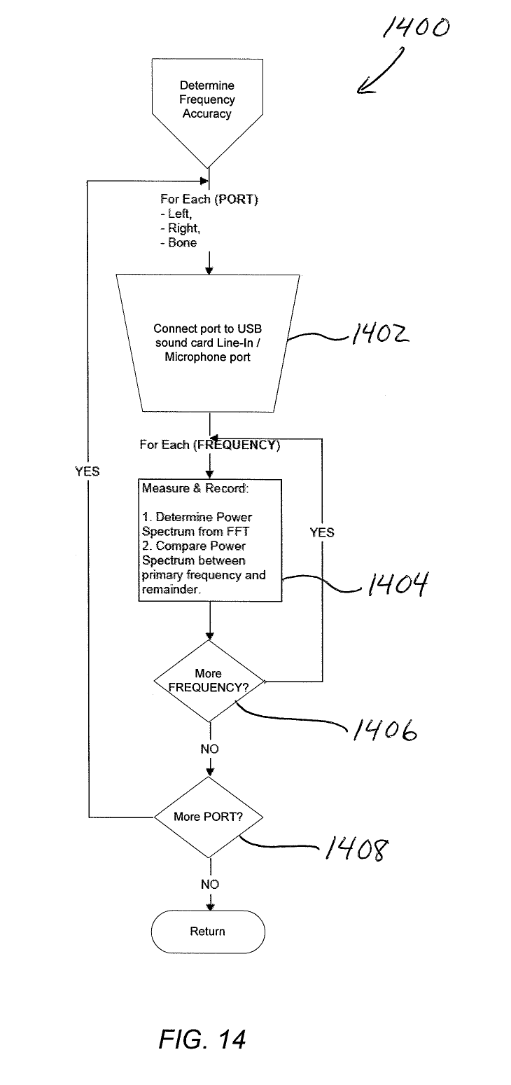

According to some embodiments, the audiometric testing device can calibrate one or more electroacoustic features in addition to pressure/force levels, including for example, attenuator linearity, frequency, and harmonic distortion. Turning to FIG. 14, a flow diagram is shown illustrating a method 1400 of analyzing frequency characteristics of pure tone outputs. Analyzing the frequency components of a pure tone output can in some cases allow for fine tuning the frequency output in order to, e.g., decrease harmonic distortion. According to some embodiments, an audio output (e.g., the auxiliary output 322 shown in FIG. 4) is coupled to an external computing device such as a PC to analyze the signal. In some embodiments a processor within the audiometric testing device may perform the frequency analysis.

In the embodiment shown in FIG. 14, the method 1400 includes selecting a tone output port and coupling 1402 the port to an external sound card, such as an internal or external sound card commonly used with a PC, with an appropriate cable. For each generated frequency, a frequency analysis of the signal is conducted 1404 by the sound card and/or associated processing components and the measured results are compared to the expected results. For example, in some cases a Fast Fourier Transform is computed for the signal to determine frequency power spectrum. Thus differences between the fundamental frequency and its harmonics can be determined and appropriate actions can be implemented if, e.g., harmonic distortion is above a desired threshold or the fundamental frequency is outside a desired tolerance range. In some embodiments in which the audiometric testing device does not meet expected performance levels, the device may notify an operator that the device should be sent back to the factory or other party for repair or replacement.

After analyzing a single frequency, the method 1400 determines 1406 whether analysis of additional frequencies is desired and the frequency analysis 1404 is repeated if necessary. After analyzing all the desired frequencies, it is determined 1408 whether a frequency analysis is desired for one or more additional testing transducers, and the method can be repeated for each additional transducer. After ending the calibration routine, the method may then prompt the operator to (or may automatically) save the analyses and/or take one or more actions to address any issues related to tone frequency.

According to some embodiments, an audiometric testing device may also monitor background or environmental noise through the calibration transducer such as a microphone, or through a separate, attached microphone (e.g., a microphone used to record patient responses during speech audiometry). In certain cases, the testing device and/or coupled external computing device may monitor the level of background noise to determine if it may affect the accuracy of audiometric tests. For example, a processor may be programmed to determine if the background noise rises above a predetermined threshold. In such a case the testing device may take a one of a number of actions, including warning the patient/technician that the noise level is high and/or pausing or stopping a hearing test. In some cases the background noise level may also be monitored before, during, or after a calibration procedure to determine if background noise may affect the calibration of the testing device.

As is known, audiometers must usually be calibrated at least once a year in most states and have their calibration traceable to the National Bureau of Standards (NBS). As previously discussed, embodiments of the invention advantageously allow calibration of the audiometric testing device at any time without the need for expensive, complex stand-alone calibration instruments. Normally, stand-alone calibration devices are tested and certified to meet the NBS calibration requirements by sending each stand-alone calibration device to a calibration laboratory that is equipped to test the calibration device and certify its performance.

As will be appreciated, periodically sending an audiometric testing device to a calibration laboratory to test and certify its calibration functionality is less than ideal. Doing so can be expensive and limits the availability of the testing device for hearing testing in the meantime. Thus, in some embodiments, methods and devices are provided for a more portable manner of ensuring the testing device meets NBS standards.

For example, according to some embodiments, a portable calibration device may be provided that emits a calibrated pure tone at each frequency to be used in the audiometric testing device. In some cases the calibration device (e.g., the size of a softball) would fit onto the calibration coupler of the testing device and could be used to “teach” the testing device what a pure tone at a specified level sounds like. Preferably, the calibration device would be a modest cost device that can be sent out annually to a calibration laboratory and calibrated to NBS. By using such a device, traceability to NBS can be ensured with a certificate of annual calibration on the calibration device.

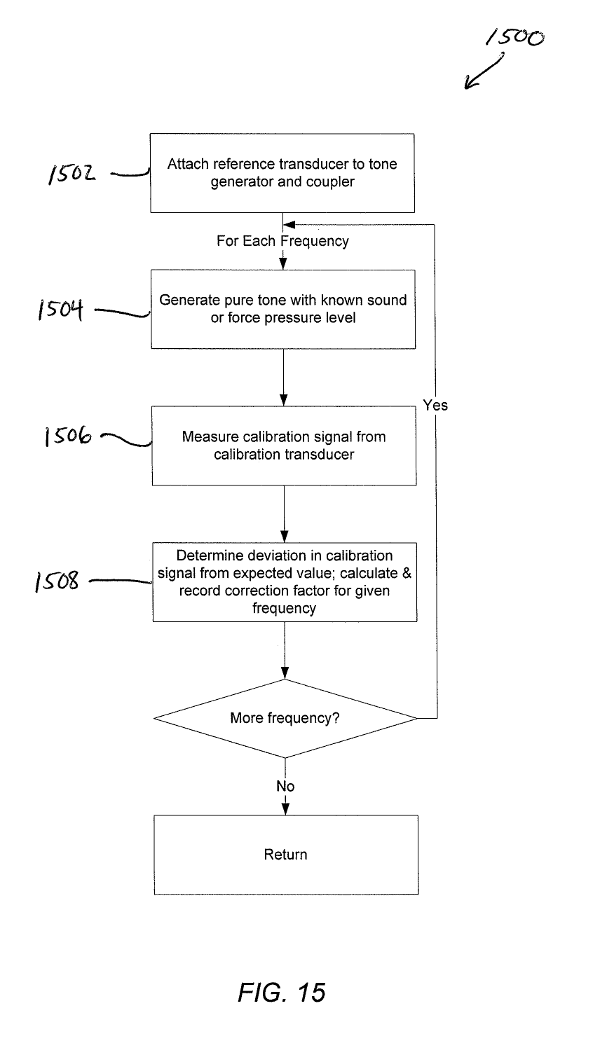

Turning to FIG. 15, a method 1500 of using a reference transducer to determine the calibration accuracy of an audiometric testing device is shown in accordance with an embodiment of the invention. The reference transducer can be any standard testing transducer, such as an earphone or a bone vibrator that has a known input-output transfer function. For example, the transfer function of a reference earphone may be determined using a standard sound level meter, such as those available from Brüel & Kjær. The method includes connecting the reference transducer to the tone generator (i.e., tone output) and attaching 1502 the reference transducer to a transducer coupler of an audiometric testing device. For a given frequency, a pure tone is generated 1504 with a known nominal sound or force pressure level. The output of the reference transducer is detected by the calibration transducer and converted to a calibration signal, which is then measured 1506 to determine 1508 any deviation from the expected output according to the known transfer function. Upon determining the presence of a deviation, a correction factor can be calculated and recorded for the current frequency and used to adjust the transfer function of the calibration transducer when measuring the output of one or more testing transducers.

According to some embodiments, the method 1500 may only calibrate a single frequency for the calibration transducer. Such an embodiment may be used when the calibration transducer is a high-quality transducer, such as a microphone having a substantially flat frequency response. In some embodiments for example, the calibration transducer may be a microphone having an approximately flat frequency response, e.g., +/−<1 dB. In some cases a very precise microphone may be used such as those used in sound level meters available from Brüel & Kjær and Larson Davis. Such high-quality transducers are often used in stand-alone calibration instrumentation because they provide a reliable output independent of frequency. According to some embodiments of the invention, the method 1500 may cycle through multiple frequencies of interest, thus providing a quick and automated method of profiling the transfer function of a calibration transducer that has a more frequency-dependent output. Thus, embodiments of the invention can advantageously employ calibration transducers that may be less frequency independent (and less expensive) than those used in stand-alone calibration instruments, while still ensuring that the transducer output is accurately correlated to respective sound pressure levels and/or conduction force levels. For example, in some cases a calibration transducer may have a frequency response that varies more than 1 dB over a range of selected frequencies. In some embodiments a microphone has a frequency response of between about +/−1 dB and +/−2 dB up to 8 kHz. According to some embodiments, the frequency response is about +/−6 dB up to 8 kHz. Other frequency tolerances, both larger and smaller, are also possible according to embodiments of the invention depending, e.g., upon a particular microphones dynamic range.

Thus, embodiments of the invention are disclosed. Although the present invention has been described in considerable detail with reference to certain disclosed embodiments, the disclosed embodiments are presented for purposes of illustration and not limitation and other embodiments of the invention are possible. One skilled in the art will appreciate that various changes, adaptations, and modifications may be made without departing from the spirit of the invention and the scope of the appended claims.

What is claimed is:

1. An audiometric testing device, comprising:
a housing comprising a tone output and a coupler, the tone output adapted to couple with a lead of a testing transducer and the coupler adapted to receive the testing transducer;
a tone generator positioned within the housing and coupled to the tone output, the tone generator adapted to generate a plurality of tones in a testing sequence and a plurality of tones in a calibration sequence;
a calibration transducer positioned proximate to the coupler and adapted to convert an output of the testing transducer to a calibration signal; and
a signal measurement module positioned within the housing, the signal measurement module coupled to the calibration transducer and adapted to measure the calibration signal resulting in a calibration measurement for adjusting operation of the tone generator.

2. The audiometric testing device of claim 1, wherein the coupler is an earphone coupler or a bone vibrator coupler.

3. The audiometric testing device of claim 1, wherein the tone output is adapted to couple to an external computing device for analyzing frequency characteristics of one or more tones.

4. The audiometric testing device of claim 1, further comprising a processor positioned within the housing and coupled to the tone generator and the signal measurement module, the processor adapted to receive calibration measurements from the signal measurement module and provide tone generation instructions to the tone generator.

5. The audiometric testing device of claim 4, wherein the housing further comprises a communication port coupled to the processor, the communication port adapted to couple the processor with an external computing device.

6. The audiometric testing device of claim 1, wherein the housing further comprises a communication port coupled to the tone generator and the signal measurement module, the communication port adapted to couple the tone generator and the signal measurement module with an external computing device.

7. An audiometric testing system, comprising the audiometric testing device of claim 6 and an external computing device coupled to the communication port, the external computing device having a processor and a non-transitory computer-readable storage medium comprising instructions for causing the processor to control the audiometric testing device based in part on inputs received through the external computing device.

8. An audiometric testing system, comprising the audiometric testing device of claim 1 and an earphone adapted to couple to the tone output and the coupler, wherein the earphone has a known input-output transfer function for calibrating the calibration transducer.

9. An audiometric testing system, comprising:
one or more processors;
a tone generator coupled with the one or more processors, the tone generator adapted to generate a plurality of tones in a testing sequence and a plurality of tones in a calibration sequence based on instructions from the one or more processors;
a tone output coupled to the tone generator and adapted to couple with a lead of a testing transducer;
a coupler adapted to receive the testing transducer;
a calibration transducer positioned proximate the coupler and adapted to convert an output of the testing transducer to a calibration signal; and
a signal measurement module coupled to the calibration transducer and the one or more processors, the signal measurement module adapted to measure the calibration signal resulting in a calibration measurement and transmit the calibration measurement to the one or more processors for adjusting operation of the tone generator based on the calibration measurement.

10. The audiometric testing system of claim 9, further comprising a housing comprising the tone generator, the tone output, the coupler, the calibration transducer, the signal measurement module, and a communication port, and an external computing device removably coupled to the communication port, the external computing device comprising at least one of the one or more processors.

11. The audiometric testing system of claim 10, wherein the external computing device further comprises a non-transitory computer-readable storage medium comprising instructions for causing the at least one processor of the external computing device to control the tone generator based in part on inputs received through the external computing device.

12. The audiometric testing system of claim 11, wherein the tone output is adapted to couple to the external computing device for analyzing frequency characteristics of one or more tones.

13. The audiometric testing system of claim 9, further comprising a non-transitory computer-readable storage medium comprising instructions for causing at least one of the one or more processors to adjust at least one of the plurality of tones generated by the tone generator based on the calibration measurement.

14. The audiometric testing system of claim 9, wherein the coupler is an earphone coupler or a bone vibrator coupler.

15. A method for calibrating an audiometric testing device, comprising:
generating a sequence of tones with the audiometric testing device and outputting the sequence of tones through a testing transducer coupled to the audiometric testing device;
detecting with the audiometric testing device a sequence of outputs from the testing transducer corresponding to the sequence of tones;
generating with the audiometric testing device calibration signals based on the sequence of outputs;
measuring the calibration signals with the audiometric testing device to generate corresponding calibration measurements; and
adjusting the generation of one or more tones based on the calibration measurements.

16. The method of claim 15, further comprising adjusting an intensity of the one or more tones based on the calibration measurements.

17. The method of claim 15, further comprising measuring a frequency characteristic of one or more tones.

18. The method of claim 15, further comprising sending the calibration measurements to an external computing device coupled to the audiometric testing device and receiving instructions from the external computing device for generating the sequence of tones and corresponding adjustments.

19. The method of claim 15, further comprising generating a sequence of calibration tones with the audiometric testing device and outputting the sequence of calibration tones through a reference transducer coupled to the audiometric testing device, the reference transducer having a known input-output transfer function for calibrating a calibration transducer of the audiometric testing device.

20. The method of claim 15, further comprising displaying instructions for guiding a user through one or more manual steps in the method.
